(19) United States
(12) Patent Application Publication
(10) Pub. No.: US 2005/0182317 A1
(43) Pub. Date: Aug. 18, 2005

(54) Method and apparatus for locating medical devices in tissue

(76) Inventor: Souheil F. Haddad, Bloomington, IN (US)

Correspondence Address: BARNES & THORNBURG, 11 SOUTH MERIDIAN, INDIANAPOLIS, IN, 46204, US

(21) Appl. No.: 11/032,760

(22) Filed: Jan. 11, 2005

Related U.S. Application Data
(60) Provisional application No. 60/540,306, filed on Jan. 29, 2004.
(60) Provisional application No. 60/554,795, filed on Mar. 19, 2004.
(60) Provisional application No. 60/567,487, filed on May 3, 2004.

(51) Int. Cl. A61B 5/05

(52) U.S. Cl. 600/424

(57) ABSTRACT

An apparatus for determining a trajectory for insertion of a medical device into a patient includes a first surface and a second surface which are radiolucent and transparent or translucent. The second surface can be spaced from the first surface so that a point on the first surface and a point on the second surface define the trajectory. Alternatively, a surface which is radiolucent and transparent or translucent can be spaced from the patient so that a point on the surface and a point on the patient define the trajectory. A method for determining a trajectory for insertion of a medical device into tissue comprises visualizing the tissue using at least one of x-ray exposures and fluoroscopy, determining the trajectory for insertion of the medical device into the tissue using the apparatus, and inserting the medical device into the tissue along the determined trajectory.

### CROSS REFERENCE TO RELATED APPLICATIONS

This regular utility patent application claims the benefit under 35 U. S. C. §119(e) of the filing dates of U.S. Ser. No. 60/540,306 filed Jan. 29, 2004, U.S. Ser. No. 60/554,795 filed Mar. 19, 2004, and U.S. Ser. No. 60/567,487 filed May 3, 2004. The disclosures of all of these prior applications are hereby incorporated herein by reference.

### FIELD OF THE INVENTION

This invention relates to methods and apparatus for locating medical devices in tissue. It is disclosed in the context of methods and apparatus for locating pedicle screws in pedicles. However, it is believed to be useful in other applications as well.

### BACKGROUND OF THE INVENTION

Various methods and devices are known for locating and affixing pedicle screws in pedicles. There are, for example, the methods and devices illustrated and described in U.S. patents and published patent applications: U.S. Pat. Nos. 6,645,204; 6,638,281; 6,638,276; 6,610,065; 6,605,095; 6,579,244; 6,569,164; 6,562,046; 6,547,795; 6,546,279; 6,546,277; 6,533,790; 6,529,765; 6,477,400; 6,470,207; 6,379,354; 6,351,662; 6,336,927; 6,298,262; 6,287,313; 6,285,902; 6,282,437; 6,273,896; 6,198,794; 6,190,320; 6,174,311; 6,069,932; 6,019,759; 6,017,343; 6,010,504; 5,904,682; 5,799,055; 5,772,594; 5,730,754; 5,716,357; 5,607,425; 5,562,695; 20030220689; 20030199882; 20030187351; 20030187348; 20030181919; 20030078495; 20030073901; 20030032965; 20020077632; 20020054662; 20020016592; 20010036245; and, 20010027320. This listing is not intended as a representation that a thorough search of all material prior art has been conducted, or that no more pertinent prior art exists, or that the listed references are material. Nor should any such representation be inferred.

Bone and connective tissue damage can result from conditions such as trauma or deterioration of, or damage to, bone or connective tissue due to genetic disorders, developmental disorders, chronic stress, aging, disease, and the like. Exemplary pathologies where bone and connective tissue damage occur include scoliosis, kyphosis, lordosis, spondylolisthesis, osteoporosis, pseudoarthrosis, spinal stenosis, and any other type of damage, deterioration, or injury to bone or connective tissue that requires orthopedic surgical intervention. Spinal column damage or damage to other bones or connective tissue can limit the range of motion of joints and can make critical elements of the nervous system vulnerable to injury.

Accordingly, a variety of surgical techniques and systems have been developed to treat damaged bone and connective tissue. Such surgical techniques include implantation of devices that consist of rods, plates, or screws, or a combination of these devices, for attachment to bones to align bones or to fuse bones by immobilization. Vertebroplasty is another such surgical technique and is used to treat fractures, such as compression fractures resulting from spinal bone loss. Vertebroplasty is a minimally invasive procedure that involves the injection of a contrast dye into the fracture followed by injection of a cement-like substance through a needle using fluoroscopy to monitor the flow of the cement-like substance into the fracture.

A typical surgical technique used to immobilize the spinal column is the implantation of rods adjacent the spinal column. The rods are attached to the spinal column by screws. This technique can be used to treat scoliosis, kyphosis, lordosis, spondylolisthesis, osteoporosis, pseudoarthrosis, spinal stenosis, and the like. The screws that are used to attach such rods, or other devices such as plates, to the spinal column are typically inserted into the pedicles. The pedicles are two dense, stem-like structures that extend, one from each side of the vertebrae on the posterior side of the vertebrae. The pedicles are the strongest part of the vertebrae, and thus are used as anchors for the screws (called pedicle screws) that are used to attach such rods or other implanted devices to the spinal column.

Because of the proximity of the spinal cord and its branching nerves, insertion of pedicle screws into the spinal column is closely monitored using, for example, fluoroscopy or x-ray exposures, or a combination of these techniques, so that the pedicle screws are inserted into the pedicles along trajectories that are alignment with the pedicles' axes. Technologically advanced systems such as the StealthStation™ Treatment Guidance System and the FluoroNav™ Virtual Fluoroscopy System (Medtronic Sofamor Danek), and other systems, assist surgeons, using real-time monitoring, in determining the proper trajectory and depth for inserting pedicle screws and other medical devices such as needles. However, such systems expose both the patient and the physician to radiation, so it is important to minimize the time that is required for determination of the proper trajectory(ies) of (a) pedicle screw(s), and get the pedicle screw(s) installed along this (these) trajectory(ies). Accordingly, there is a need for apparatus and methods to assist physicians in determining the proper trajectory for insertion of a pedicle screw or other medical device used for orthopedic applications, into a patient while reducing the exposure of both the patient and the physician to radiation.

### DISCLOSURE OF THE INVENTION

According to a first aspect of the invention, an apparatus for determining a trajectory for insertion of a medical device into tissue includes a first surface which is radiolucent and transparent or translucent and a second surface which is radiolucent and transparent or translucent. The second surface is spaced from the first surface so that a point on the first surface and a point on the second surface define the trajectory.

Further illustratively according to this aspect of the invention, the apparatus comprises means for immobilizing the first and second surfaces relative to the tissue.

Illustratively according to this aspect of the invention, the first and second surfaces are substantially parallel.

Alternatively illustratively according to this aspect of the invention, the first and second surfaces are not substantially parallel.

Illustratively according to this aspect of the invention, a distance between the first and second surfaces is adjustable.

Additionally illustratively according to this aspect of the invention, the first and second surfaces are separated by a distance sufficient to permit entry of an instrument between the first and second surfaces to mark at least one of the first and second surfaces.

Illustratively according to this aspect of the invention, the medical device is selected from the group consisting of a wire, a needle, a tap, a screw, a hook, a pin, a staple, a depth gauge, a drill, a drill guide, a probe, a device useful for orthopedic surgical intervention, and combinations of these.

Further illustratively according to this aspect of the invention, at least one of the first and second surfaces comprises a plastic.

Additionally illustratively according to this aspect of the invention, the at least one of the first and second surfaces comprises a plastic selected from the group consisting of acrylic, epoxy, polyester, polypropylene, polyurethane, polyethylene, polycarbonate, polystyrene, polysulfone, polyetherimide, polyethersulfone, polyphenylsulfone, polyphenylsulfide, acrylonitrile-butadiene-styrene polymer, polyetheretherketone, and combinations thereof.

Illustratively according to this aspect of the invention, the at least one of the first and second surfaces comprises a filler.

According to another aspect of the invention, a method for determining a trajectory for insertion of a medical device into tissue comprises visualizing the tissue using at least one of x-ray exposures and fluoroscopy, determining the trajectory for insertion of the medical device into the tissue using the apparatus of the first aspect of the invention, and inserting the medical device into the tissue along the determined trajectory.

Further illustratively according to this aspect of the invention, the method comprises immobilizing the first and second surfaces relative to the tissue.

Further illustratively according to this aspect of the invention, the method comprises orienting the second surface at a distance from the first surface.

Illustratively according to this aspect of the invention, orienting the second surface at a distance from the first surface comprises orienting the second surface at a distance from the first surface sufficient to permit entry of an instrument between the first and second surfaces to mark at least one of the first and second surfaces

Illustratively according to this aspect of the invention, determining a trajectory for insertion of a medical device into tissue comprises determining a trajectory for insertion of a medical device selected from the group consisting of a wire, a needle, a tap, a screw, a hook, a pin, a staple, a depth gauge, a drill, a drill guide, a probe, a device useful for orthopedic surgical intervention, and combinations of these, into tissue.

According to another aspect of the invention, a method for determining a trajectory for insertion of a medical device into tissue comprises providing a first surface which is radiolucent and transparent or translucent, providing a second surface which is radiolucent and transparent or translucent, spacing the second surface from the first surface and marking a first point on the first surface and a second point on the second surface to define the trajectory.

Further illustratively according to this aspect of the invention, the method comprises immobilizing the first and second surfaces relative to the tissue.

Illustratively according to this aspect of the invention, spacing the second surface from the first surface comprises orienting the second surface so that it is substantially parallel with the first surface.

Additionally illustratively according to this aspect of the invention, spacing the second surface from the first surface comprises orienting the second surface so that it is not substantially parallel with the first surface.

Illustratively according to this aspect of the invention, spacing the second surface from the first surface comprises adjustably spacing the second surface from the first surface.

Further illustratively according to this aspect of the invention, spacing the second surface from the first surface comprises spacing the second surface from the first surface a distance sufficient to permit entry of an instrument between the first and second surfaces to mark at least one of the first and second surfaces.

Additionally illustratively according to this aspect of the invention, determining a trajectory for insertion of a medical device into tissue comprises determining a trajectory for insertion of a medical device selected from the group consisting of a wire, a needle, a tap, a screw, a hook, a pin, a staple, a depth gauge, a drill, a drill guide, a probe, a device useful for orthopedic surgical intervention, and combinations of these, into tissue.

Illustratively according to this aspect of the invention, providing a first surface which is radiolucent and transparent or translucent and providing a second surface which is radiolucent and transparent or translucent together comprise providing at least one of the first and second surfaces constructed from plastic.

Further illustratively according to this aspect of the invention, providing at least one of the first and second surfaces constructed from plastic comprises providing at least one of the first and second surfaces constructed from plastic selected from the group consisting of acrylic, epoxy, polyester, polypropylene, polyurethane, polyethylene, polycarbonate, polystyrene, polysulfone, polyetherimide, polyethersulfone, polyphenylsulfone, polyphenylsulfide, acrylonitrile-butadiene-styrene polymer, polyetheretherketone, and combinations thereof.

Additionally illustratively according to this aspect of the invention, providing at least one of the first and second surfaces constructed from plastic comprises providing at least one of the first and second surfaces constructed from filled plastic.

### BRIEF DESCRIPTION OF THE DRAWINGS

The invention may best be understood by referring to the following detailed description and accompanying drawings which illustrate the invention. In the drawings:

FIG. 1 illustrates a fragmentary sectional rear elevational view of a vertebra illustrating the locations of the pedicles, vertebral body, and spinal cord, and illustrating a proper trajectory for insertion of a medical device, such as a pedicle screw;

FIG. 2 illustrates a fragmentary sectional side elevational view of a vertebra illustrating the locations of the pedicles, vertebral body, and spinal cord, and illustrating a proper trajectory for insertion of a medical device, such as a pedicle screw;

FIG. 3 illustrates a fragmentary sectional top plan view of a vertebra illustrating the locations of the pedicles, vertebral body, and spinal cord, and illustrating a trajectory for insertion of a medical device, such as a pedicle screw;

FIG. 4 illustrates a perspective view of an apparatus constructed according to an aspect of the invention illustrating a trajectory for insertion of a medical device;

FIG. 5 illustrates a perspective view of an apparatus constructed according to an aspect of the invention illustrating a trajectory for insertion of a medical device;

FIG. 6 illustrates a perspective view of an apparatus constructed according to an aspect of the invention illustrating a trajectory for insertion of a medical device;

FIG. 7 illustrates a perspective view of an apparatus constructed according to an aspect of the invention illustrating a trajectory for insertion of a medical device;

FIG. 8 illustrates a perspective view of an apparatus constructed according to an aspect of the invention illustrating a trajectory for insertion of a medical device;

FIG. 9 illustrates a perspective view of an apparatus constructed according to an aspect of the invention illustrating a trajectory for insertion of a medical device;

FIG. 10 illustrates a perspective view of an apparatus constructed according to an aspect of the invention illustrating a trajectory for insertion of a medical device;

FIG. 11 illustrates a perspective view of an apparatus constructed according to an aspect of the invention illustrating a trajectory for insertion of a medical device;

FIG. 12 illustrates a perspective view of an apparatus constructed according to an aspect of the invention illustrating a trajectory for insertion of a medical device; and

FIG. 13 illustrates a perspective view of an apparatus constructed according to an aspect of the invention illustrating a trajectory for insertion of a medical device.

### DETAILED DESCRIPTIONS OF ILLUSTRATIVE EMBODIMENTS

As used in this application, “trajectory” means a path along which a device can be inserted into human or animal tissue.

As used in this application, “translucent” means permitting the passage of light.

As used in this application, “transparent” means permitting the passage of light without appreciable light scattering.

As used in this application, “radiolucent” means partly or wholly permeable to radiation.

As used in this application, “plastic” includes, but is not limited to, natural and synthetic resins and polymers.

Systems, such as fluoroscopy, have been developed to assist healthcare workers, using real-time monitoring or substantially real-time monitoring, in determining the proper trajectories and depths for inserting medical devices into tissues to avoid damage to adjacent tissues. However, such systems can require lengthy exposure of both patients and healthcare workers to radiation. Accordingly, there is a need for an apparatus that can be used to assist a healthcare worker in determining the proper trajectory for insertion of a medical device into a patient while reducing the exposure of both the patient and the healthcare worker to radiation. The invention disclosed herein relates to an apparatus and method for determining the trajectory for insertion of a medical device into a patient.

Illustratively, the apparatus and methods of the invention can be used for any type of intervention, including implanting rods, plates, screws, hooks, pins, staples, and the like, or combinations of these, to attach, align, stabilize or fuse bones and the like. These techniques typically require the insertion of such devices as screws, hooks, pins and staples into bones while avoiding injury to, for example, surrounding tissue. The apparatus and method of the invention can be used to determine the trajectory for insertion of a medical device, such as a screw, pin, wire, needle, tap, depth gauge, drill, drill guide, probe, burr, awl, or the like, while avoiding injury to, for example, surrounding tissue or nerves.

Illustratively, the apparatus and method described herein can also be used for vertebroplasty which is a surgical technique used to treat fractures, such as compression fractures resulting from spinal bone loss. Vertebroplasty involves the injection of a contrast dye into a fracture followed by injection of a cement-like substance through a needle using fluoroscopy to monitor the flow of the cement-like substance into the fracture. The apparatus and method of the invention can be used to determine the trajectory for insertion of the needle into the compression fracture to avoid injury to surrounding nerves or to the spinal cord. Illustratively, the apparatus and method of the invention can also be used in any other type of surgical technique that requires insertion of a needle along a determined trajectory.

Illustratively, the apparatus and method of the invention can be used to treat any disorder that requires orthopedic surgical intervention such as, for example, scoliosis, kyphosis, lordosis, spondylolisthesis, osteoporosis, pseudoarthrosis, spinal stenosis, and to stabilize osteotomies, to treat fractures, and to treat any type of connective tissue damage (e.g., connective tissue damage requiring arthroscopic surgery).

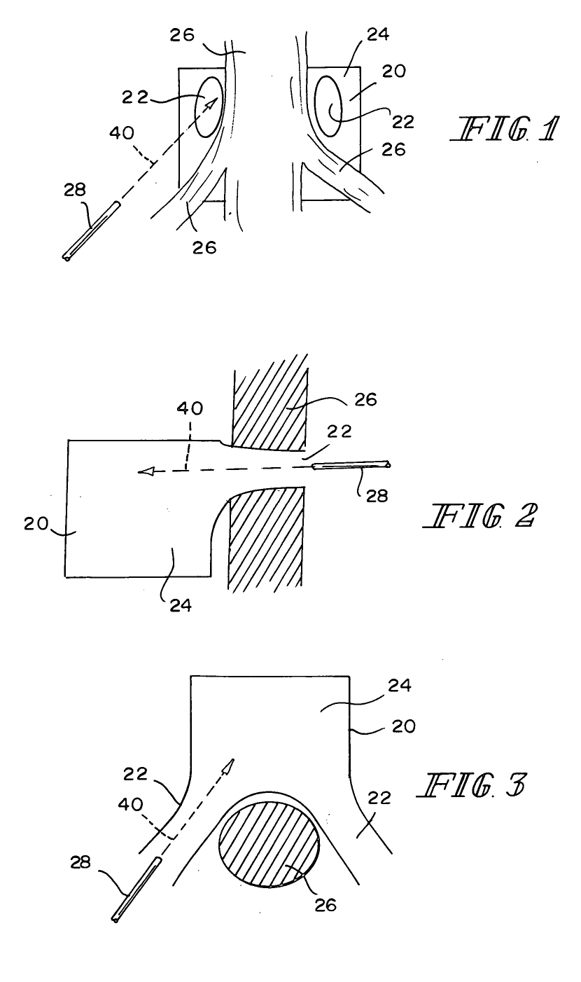

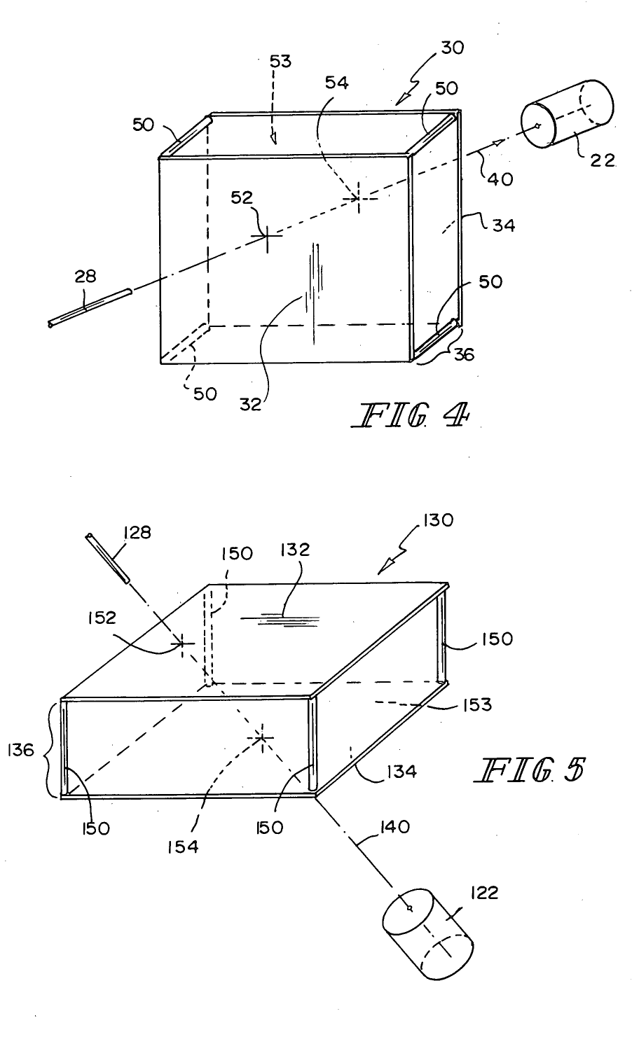

The invention is applicable to any surgical technique in which it would be useful to determine the trajectory for insertion of a medical device into a patient. However, the apparatus and method of the invention are disclosed in the context of determining the trajectory for the insertion of pedicle screws into the spinal column. FIGS. 1-3 illustrate views of vertebrae 20 and show the locations of the pedicles 22, vertebral body 24, and spinal cord and nerve tissue 26, and illustrate appropriate trajectories 40 for insertion of a medical device 28, such as a K-wire, into a pedicle 22.

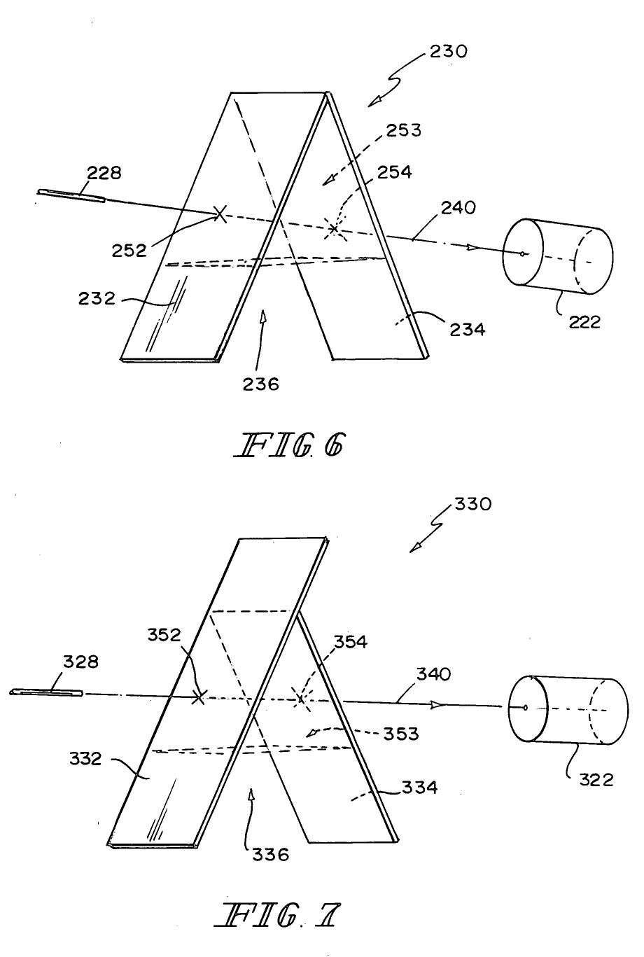

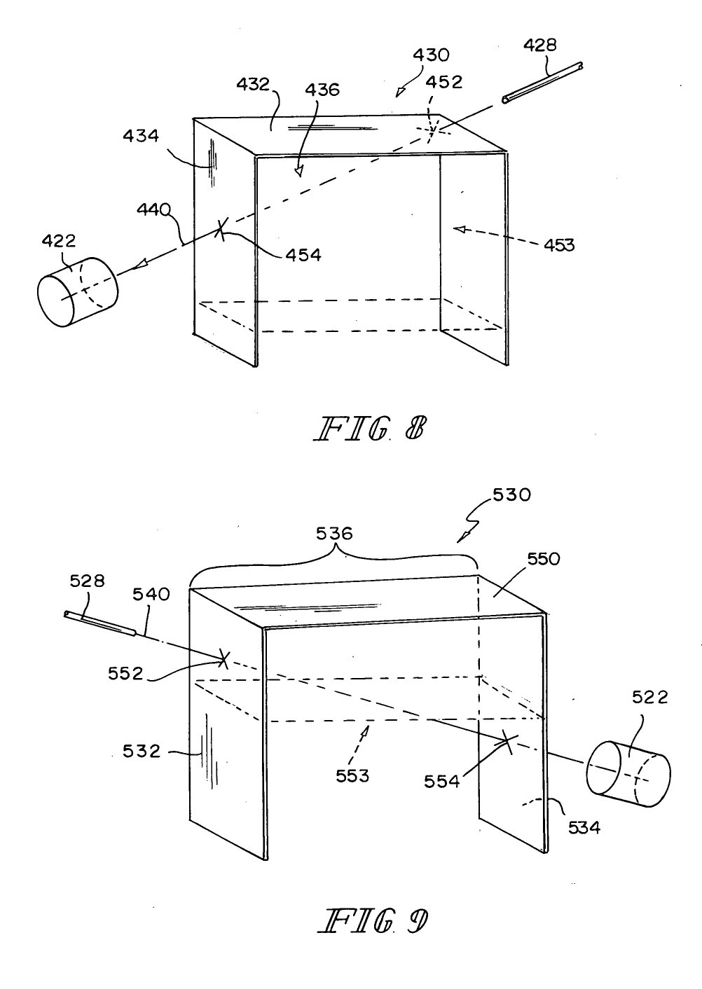

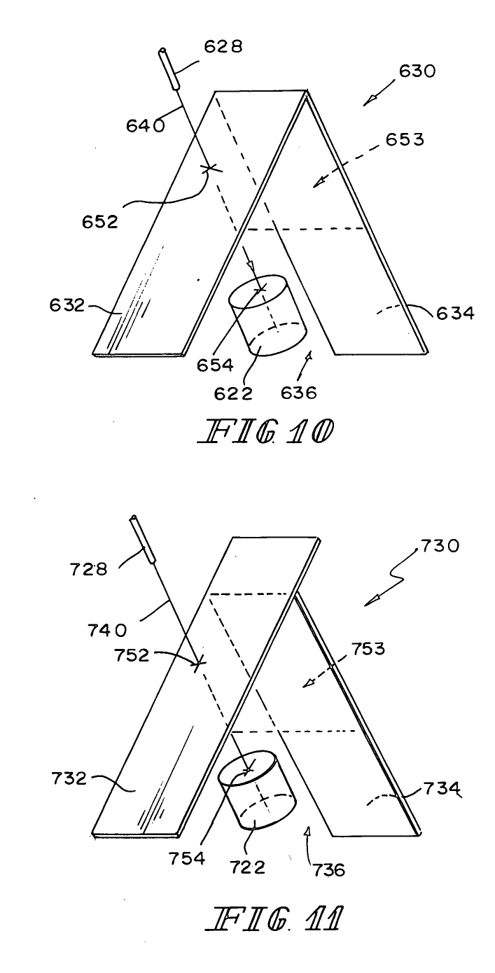

Referring now to FIG. 4, an apparatus 30 comprises a first surface 32 and a second surface 34. The first and second surfaces 32, 34 are separated by a distance 36. Insertion of a medical device 28, such as, for example, a K-wire, through the first surface 32 and the second surface 34 along a trajectory 40 and into, for example, a pedicle 22 along the determined trajectory 40 will appropriately locate the medical device 28.

The first and second surfaces 32, 34 are radiolucent to permit the passage of radiation through the first and second surfaces 32, 34 and into the patient to permit visualization of the pedicle 22. The first and second surfaces 32, 34 desirably are also transparent or translucent to permit the passage of light to facilitate determination of the trajectory 40 for insertion of, for example, a K-wire 28 into a pedicle 22 as described in more detail below.

The first and second surfaces 32, 34 are coupled together by adjustable or non-adjustable rods 50 (FIG. 4), bolts, clamps, posts, plates, or any other type of support that separates the first and second surfaces 32, 34 and holds the first and second surfaces 32, 34 in spatially separated, fixed positions in relation to each other while the apparatus 30 is in use. One or both surfaces 32, 34 can be used to steady and/or hold the instrument 28 being inserted in the pedicle 22 while further adjustments are made to the trajectory 40 under fluoroscopy or other types of guidance, such as stealth navigation, or the like.

Illustratively, the first and second surfaces 32, 34 can be, for example, plastic, such as, for example, acrylic, epoxy, polypropylene, polyurethane, polyethylene, polycarbonate, polystyrene, polysulfone, polyetherimide, polyethersulfone, polyphenylsulfone, polyphenylsulfide, acrylonitrile-butadiene-styrene, polyetheretherketone, or combinations thereof. The plastic can be unfilled or can be filled with, for example, glass fiber, graphite fiber, or any other suitable filler. The type of plastic and absence or presence and amount of filler typically will be determined by, for example, the required strength and/or rigidity of the apparatus 30, its required translucence or transparency, its radiolucence, and so on. The plastic can be any type of plastic known in the art that meets the requirements of a particular application for radiolucence, translucence or transparency, strength, rigidity, and so on. Typically, the plastic will need to be sufficiently rigid and strong to withstand being perforated using a drill bit, a sharp needle, or other device 28, to form aligned holes in the first and second surfaces 32, 34 to define the trajectory 40. Additionally, the space between the two surfaces 32, 34 may be partly or completely filled with a radiolucent material 53, such as a resin foam or the like, that can enhance the structural rigidity of the apparatus 30 and further help steady the instrument 28. This is illustrated in broken lines in FIG. 4.

The first and second surfaces 32, 34 can be substantially parallel, as illustrated in the embodiments of FIGS. 4-5, 9, and 12 or can intersect, as illustrated in the embodiments of FIGS. 6-7. The first and second surfaces 32, 34 can be spatially oriented in any way that facilitates determination of a suitable trajectory 40 for insertion of a medical device 28.

Illustratively, the first 32 and second surfaces 34 are separated by a distance 36 that facilitates determination of a suitable trajectory 40 to be determined for insertion of (a) medical device(s) 28, such as a K-wire followed by a pedicle screw, into the tissue 22. In some embodiments, the spacing 36 between the first and second surfaces 32, 34 is determined during the construction of the apparatus 30. In other embodiments, the distance 36 between the first and second surfaces 32, 34 is established as the apparatus 30 is set up for use. In embodiments in which the separation 36 of the first and second surfaces 32, 34 is predetermined, the length of each device 50 used to connect the first and second surfaces 32, 34 can be fixed. In embodiments in which the separation of the first and second surfaces 32, 34 is established as the apparatus 30 is set up for use, devices 50 used to connect the first and second surfaces 32, 34 are adjustable or are available to the user in different lengths from which the user selects appropriate lengths during setup.

The apparatus 30 is immobilized (that is, fixed to the operating table or any other stationary equipment in the operating room) at a desired location relative to the tissue 22. The anatomy of the tissue 22 is then visualized by using such techniques as x-ray exposures or fluoroscopy to establish the desired trajectory 40. A suitable radiopaque device 28, such as, for example, a K-wire, is then used to locate the points 52, 54 at which the trajectory 40 passes through, for example, the first and second surfaces 32, 34. The points 52, 54 are then marked, for example, with a sterile marking pen or by scratching an index with a sterile scribe or the like. The trajectory between points 52, 54 is the desired trajectory 40 into and through the tissue 22.

Instrument 28, can also be used to form holes at points 52, 54. Instrument 28 can then be advanced, for example, through the skin and soft tissue of the patient to enter the pedicle 22 along the appropriate trajectory 40. Alternatively, (an)other device(s) can be used to form holes at points 52, 54. A K-wire, for example, can be used to mark the desired trajectory 40 and can then be used to guide other instruments such as a probe, a tap, a drill, a pedicle screw, and so on, along the determined trajectory 40 into the tissue 22.

Referring now to FIG. 5, an apparatus 130 comprises a first surface 132 and a second surface 134. The first and second surfaces 132, 134 are separated by a distance 136. Insertion of a medical device 128, such as, for example, a K-wire, through the first surface 132 and the second surface 134 along a trajectory 140 and into, for example, a pedicle 122 along the determined trajectory 140 will appropriately locate the medical device 128.

The first and second surfaces 132, 134 are radiolucent to permit the passage of radiation through the first and second surfaces 132, 134 and into the patient to permit visualization of the pedicle 122. The first and second surfaces 132, 134 desirably are also transparent or translucent to permit the passage of light to facilitate determination of the trajectory 140 for insertion of, for example, a K-wire 128 into a pedicle 122 as described in more detail below.

The first and second surfaces 132, 134 are coupled together by adjustable or non-adjustable rods 150, bolts, clamps, posts, plates, or any other type of support that separates the first and second surfaces 132, 134 and holds the first and second surfaces 132, 134 in spatially separated, fixed positions in relation to each other while the apparatus 130 is in use. Additionally, the space between the two surfaces 132, 134 may be partly or completely filled with a radiolucent material 153, such as a resin foam or the like, that can enhance the structural rigidity of the apparatus 130 and further help steady the instrument 128. This is illustrated in broken lines in FIG. 5.

The apparatus 130 is immobilized (that is, fixed to the operating table or any other stationary equipment in the operating room) at a desired location relative to the tissue 122. The anatomy of the tissue 122 is then visualized by using such techniques as x-ray exposures or fluoroscopy to establish the desired trajectory 140. A suitable radiopaque device 128, such as, for example, a K-wire, is then used to locate the points 152, 154 at which the trajectory 140 passes through, for example, the first and second surfaces 132, 134. The points 152, 154 are then marked, for example, with a sterile marking pen or by scratching an index, such as a cross or the like, with a sterile scribe or the like. The trajectory between points 152, 154 is the desired trajectory 140 into and through the tissue 122.

Instrument 128, can also be used to form holes at points 152, 154. Instrument 128 can then be advanced, for example, through the skin and soft tissue of the patient to enter the pedicle 122 along the appropriate trajectory 140. Alternatively, (an)other device(s) can be used to form holes at points 152, 154. A K-wire, for example, can be used to mark the desired trajectory 140 and can then be used to guide other instruments such as a probe, a tap, a drill, a pedicle screw, and so on, along the determined trajectory 140 into the tissue 122.

Referring now to FIG. 6, an apparatus 230 comprises a first surface 232 and a second surface 234. The first and second surfaces 232, 234 intersect to define between them a space 236. Insertion of a medical device 228, such as, for example, a K-wire, through the first surface 232 and the second surface 234 along a trajectory 240 and into, for example, a pedicle 222 along the determined trajectory 240 will appropriately locate the medical device 228.

The first and second surfaces 232, 234 are radiolucent to permit the passage of radiation through the first and second surfaces 232, 234 and into the patient to permit visualization of the pedicle 222. The first and second surfaces 232, 234 desirably are also transparent or translucent to permit the passage of light to facilitate determination of the trajectory 240 for insertion of, for example, a K-wire 228 into a pedicle 222 as described in more detail below.

The first and second surfaces 232, 234 are joined together by, for example, a device such as a corner clamp or by an adhesive, or by some combination of such a device and an adhesive, that maintains the first and second surfaces 232, 234 in fixed positions in relation to each other while the apparatus 230 is in use. Additionally, the space between the two surfaces 232, 234 may be partly or completely filled with a radiolucent material 253, such as a resin foam or the like, that can enhance the structural rigidity of the apparatus 230 and further help steady the instrument 228. This is illustrated in broken lines in FIG. 6.

The apparatus 230 is immobilized (that is, fixed to the operating table or any other stationary equipment in the operating room) at a desired location relative to the tissue 222. The anatomy of the tissue 222 is then visualized by using such techniques as x-ray exposures or fluoroscopy to establish the desired trajectory 240. A suitable radiopaque device 228, such as, for example, a K-wire, is then used to locate the points 252, 254 at which the trajectory 240 passes through, for example, the first and second surfaces 232, 234. The points 252, 254 are then marked, for example, with a sterile marking pen or by scratching an index with a sterile scribe or the like. The trajectory between points 252, 254 is the desired trajectory 240 into and through the tissue 222.

Instrument 228, can also be used to form holes at points 252, 254. Instrument 228 can then be advanced, for example, through the skin and soft tissue of the patient to enter the pedicle 222 along the appropriate trajectory 240. Alternatively, (an)other device(s) can be used to form holes at points 252, 254. A K-wire, for example, can be used to mark the desired trajectory 240 and can then be used to guide other instruments such as a probe, a tap, a drill, a pedicle screw, and so on, along the determined trajectory 240 into the tissue 222.

Referring now to FIG. 7, an apparatus 330 comprises a first surface 332 and a second surface 334. The first and second surfaces 332, 334 define between them a space 336. Insertion of a medical device 328, such as, for example, a K-wire, through the first surface 332 and the second surface 334 along a trajectory 340 and into, for example, a pedicle 322 along the determined trajectory 340 will appropriately locate the medical device 328.

The first and second surfaces 332, 334 are radiolucent to permit the passage of radiation through the first and second surfaces 332, 334 and into the patient to permit visualization of the pedicle 322. The first and second surfaces 332, 334 desirably are also transparent or translucent to permit the passage of light to facilitate determination of the trajectory 340 for insertion of, for example, a K-wire 328 into a pedicle 322 as described in more detail below.

The first and second surfaces 332, 334 are joined together by, for example, a device such as a corner clamp or by an adhesive, or by some combination of such a device and an adhesive, that maintains the first and second surfaces 332, 334 in fixed positions in relation to each other while the apparatus 330 is in use. Additionally, the space between the two surfaces 332, 334 may be partly or completely filled with a radiolucent material 353, such as a structural resin foam or the like, that can enhance the structural rigidity of the apparatus 330 and further help steady the instrument 328. This is illustrated in broken lines in FIG. 7.

The apparatus 330 is immobilized (that is, fixed to the operating table or any other stationary equipment in the operating room) at a desired location relative to the tissue 322. The anatomy of the tissue 322 is then visualized by using such techniques as x-ray exposures or fluoroscopy to establish the desired trajectory 340. A suitable radiopaque device 328, such as, for example, a K-wire, is then used to locate the points 352, 354 at which the trajectory 340 passes through, for example, the first and second surfaces 332, 334. The points 352, 354 are then marked, for example, with a sterile marking pen or by scratching an index with a sterile scribe or the like. The trajectory between points 352, 354 is the desired trajectory 340 into and through the tissue 322.

Instrument 328, can also be used to form holes at points 352, 354. Instrument 328 can then be advanced, for example, through the skin and soft tissue of the patient to enter the pedicle 322 along the appropriate trajectory 340. Alternatively, (an)other device(s) can be used to form holes at points 352, 354. A K-wire, for example, can be used to mark the desired trajectory 340 and can then be used to guide other instruments such as a probe, a tap, a drill, a pedicle screw, and so on, along the determined trajectory 340 into the tissue 322.

Referring now to FIG. 8, an apparatus 430 comprises a first surface 432 and a second surface 434. The first and second surfaces 432, 434 define between them a space 436. Insertion of a medical device 428, such as, for example, a K-wire, through the first surface 432 and the second surface 434 along a trajectory 440 and into, for example, a pedicle 422 along the determined trajectory 440 will appropriately locate the medical device 428.

The first and second surfaces 432, 434 are radiolucent to permit the passage of radiation through the first and second surfaces 432, 434 and into the patient to permit visualization of the pedicle 422. The first and second surfaces 432, 434 desirably are also transparent or translucent to permit the passage of light to facilitate determination of the trajectory 440 for insertion of, for example, a K-wire 428 into a pedicle 422 as described in more detail below. Additionally, the space between the two surfaces 432, 434 may be partly or completely filled with a radiolucent material 453, such as a resin foam or the like, that can enhance the structural rigidity of the apparatus 430 and further help steady the instrument 428. This is illustrated in broken lines in FIG. 8.

The first and second surfaces 432, 434 are joined together by, for example, a device such as a corner clamp or by an adhesive, or by some combination of such a device and an adhesive, that maintains the first and second surfaces 432, 434 in fixed positions in relation to each other while the apparatus 430 is in use.

The apparatus 430 is immobilized (that is, fixed to the operating table or any other stationary equipment in the operating room) at a desired location relative to the tissue 422. The anatomy of the tissue 422 is then visualized by using such techniques as x-ray exposures or fluoroscopy to establish the desired trajectory 440. A suitable radiopaque device 428, such as, for example, a K-wire, is then used to locate the points 452, 454 at which the trajectory 440 passes through, for example, the first and second surfaces 432, 434. The points 452, 454 are then marked, for example, with a sterile marking pen or by scratching an index with a sterile scribe or the like. The trajectory between points 452, 454 is the desired trajectory 440 into and through the tissue 422.

Instrument 428, can also be used to form holes at points 452, 454. Instrument 428 can then be advanced, for example, through the skin and soft tissue of the patient to enter the pedicle 422 along the appropriate trajectory 440. Alternatively, (an)other device(s) can be used to form holes at points 452, 454. A K-wire, for example, can be used to mark the desired trajectory 440 and can then be used to guide other instruments such as a probe, a tap, a drill, a pedicle screw, and so on, along the determined trajectory 440 into the tissue 422.

Referring now to FIG. 9, an apparatus 530 comprises a first surface 532 and a second surface 534. The first and second surfaces 532, 534 are separated by a distance 536. Insertion of a medical device 528, such as, for example, a K-wire, through the first surface 532 and the second surface 534 along a trajectory 540 and into, for example, a pedicle 522 along the determined trajectory 540 will appropriately locate the medical device 528.

The first and second surfaces 532, 534 are radiolucent to permit the passage of radiation through the first and second surfaces 532, 534 and into the patient to permit visualization of the pedicle 522. The first and second surfaces 532, 534 desirably are also transparent or translucent to permit the passage of light to facilitate determination of the trajectory 540 for insertion of, for example, a K-wire 528 into a pedicle 522 as described in more detail below.

The first and second surfaces 532, 534 are joined together by, for example, a plastic sheet spacer 550, that maintains the first and second surfaces 532, 534 in fixed positions in relation to each other while the apparatus 530 is in use. Additionally, the space between the two surfaces 532, 534 may be partly or completely filled with a radiolucent material 553, such as a resin foam or the like, that can enhance the structural rigidity of the apparatus 530 and further help steady the instrument 528. This is illustrated in broken lines in FIG. 9.

The apparatus 530 is immobilized (that is, fixed to the operating table or any other stationary equipment in the operating room) at a desired location relative to the tissue 522. The anatomy of the tissue 522 is then visualized by using such techniques as x-ray exposures or fluoroscopy to establish the desired trajectory 540. A suitable radiopaque device 528, such as, for example, a K-wire, is then used to locate the points 552, 554 at which the trajectory 540 passes through, for example, the first and second surfaces 532, 534. The points 552, 554 are then marked, for example, with a sterile marking pen or by scratching an index with a sterile scribe or the like. The trajectory between points 552, 554 is the desired trajectory 540 into and through the tissue 522.

Instrument 528, can also be used to form holes at points 552, 554. Instrument 528 can then be advanced, for example, through the skin and soft tissue of the patient to enter the pedicle 522 along the appropriate trajectory 540. Alternatively, (an)other device(s) can be used to form holes at points 552, 554. A K-wire, for example, can be used to mark the desired trajectory 540 and can then be used to guide other instruments such as a probe, a tap, a drill, a pedicle screw, and so on, along the determined trajectory 540 into the tissue 522.

Referring now to FIG. 10, an apparatus 630 comprises a first surface 632 and a second surface 634. The first and second surfaces 632, 634 intersect to define between them a space 636. Surfaces 632, 634 may joined together by, for example, a device such as a corner clamp or by an adhesive, or by some combination of such a device and an adhesive, or by any other suitable mechanism(s). Insertion of a medical device 628, such as, for example, a K-wire, through surface 632 and into, for example, a pedicle 622 along a determined trajectory 640 will appropriately locate the medical device 628.

Surface 632 is radiolucent to permit the passage of radiation through surface 632 and into the patient to permit visualization of the pedicle 622. Surface 632 desirably is also transparent or translucent to permit the passage of light to facilitate determination of the trajectory 640 for insertion of, for example, a K-wire 628 into a pedicle 622 as described in more detail below. Although FIG. 10 depicts the use of surface 632 of the apparatus 630 depicted in FIG. 10 to determine the trajectory 640, surface 634 can equally as readily be used to determine the trajectory 640 in a manner similar to that described below for surface 632. Additionally, the space between the two surfaces 632, 634 may be partly or completely filled with a radiolucent material 653, such as a resin foam or the like, that can enhance the structural rigidity of the apparatus 630 and further help steady the instrument 628. This is illustrated in broken lines in FIG. 10.

The apparatus 630 is immobilized (that is, fixed to the operating table or any other stationary equipment in the operating room) at a desired location relative to the tissue 622. The anatomy of the tissue 622 is then visualized by using such techniques as x-ray exposures or fluoroscopy to establish the desired trajectory 640. A suitable radiopaque device 628, such as, for example, a K-wire, is then used to locate the points 652, 654 at which the trajectory 640 passes through, for example, the first surface 632 and into the tissue 622. The points 652, 654 are then marked, for example, with a sterile marking pen or by scratching an index with a sterile scribe or the like. The point 654 can be, for example, a point marked directly on the patient. The trajectory between points 652, 654 is the desired trajectory 640 into and through the tissue 622.

Instrument 628 can also be used to form holes at points 652, 654. Instrument 628 can then be advanced, for example, through point 654 on the patient to enter the pedicle 622 along the appropriate trajectory 640. Alternatively, (an)other device(s) can be used to form holes at points 652, 654. A K-wire, for example, can be used to mark the desired trajectory 640 and can then be used to guide other instruments such as a probe, a tap, a drill, a pedicle screw, and so on, along the determined trajectory 640 into the tissue 622.

Referring now to FIG. 11, an apparatus 730 comprises a first surface 732 and a second surface 734. The first and second surfaces 732, 734 intersect to define between them a space 736. Surfaces 732, 734 may joined together by, for example, a device such as a corner clamp or by an adhesive, or by some combination of such a device and an adhesive, or by any other suitable mechanism(s). Insertion of a medical device 728, such as, for example, a K-wire, through surface 732 and into, for example, a pedicle 722 along a determined trajectory 740 will appropriately locate the medical device 728.

Surface 732 is radiolucent to permit the passage of radiation through surface 732 and into the patient to permit visualization of the pedicle 722. Surface 732 desirably is also transparent or translucent to permit the passage of light to facilitate determination of the trajectory 740 for insertion of, for example, a K-wire 728 into a pedicle 722 as described in more detail below. Although FIG. 11 depicts the use of surface 732 of the apparatus 730 depicted in FIG. 11 to determine the trajectory 740, surface 734 can equally as readily be used to determine the trajectory 740 in a manner similar to that described below for surface 732. Additionally, the space between the two surfaces 732, 734 may be partly or completely filled with a radiolucent material 753, such as a resin foam or the like, that can enhance the structural rigidity of the apparatus 730 and further help steady the instrument 728. This is illustrated in broken lines in FIG. 11.

The apparatus 730 is immobilized (that is, fixed to the operating table or any other stationary equipment in the operating room) at a desired location relative to the tissue 722. The anatomy of the tissue 722 is then visualized by using such techniques as x-ray exposures or fluoroscopy to establish the desired trajectory 740. A suitable radiopaque device 728, such as, for example, a K-wire, is then used to locate the points 752, 754 at which the trajectory 740 passes through, for example, the first surface 732 and into the tissue 722. The points 752, 754 are then marked, for example, with a sterile marking pen or by scratching an index with a sterile scribe or the like. The point 754 can be, for example, a point marked directly on the patient. The trajectory between points 752, 754 is the desired trajectory 740 into and through the tissue 722.

Instrument 728 can also be used to form holes at points 752, 754. Instrument 728 can then be advanced, for example, through point 754 on the patient to enter the pedicle 722 along the appropriate trajectory 740. Alternatively, (an)other device(s) can be used to form holes at points 752, 754. A K-wire, for example, can be used to mark the desired trajectory 740 and can then be used to guide other instruments such as a probe, a tap, a drill, a pedicle screw, and so on, along the determined trajectory 740 into the tissue 722.

Referring now to FIG. 12, an apparatus 830 comprises first, second and third surfaces 832, 833, 834, defining among them a space 836. Insertion of a medical device 828, such as, for example, a K-wire, through one of the surfaces 832, 833, 834, and into, for example, a pedicle 822 along a determined trajectory 840 will appropriately locate the medical device 828.

Illustratively, the surfaces 832, 833, 834 are radiolucent to permit the passage of radiation through them and into the patient to permit visualization of the pedicle 822. The surfaces 832, 833, 834 desirably are also transparent or translucent to permit the passage of light to facilitate determination of the trajectory 840 for insertion of, for example, a K-wire 828 into a pedicle 822 as described in more detail below. Although FIG. 12 illustrates the use of the second surface 833 of the apparatus 830 depicted in FIG. 12 to determine the trajectory 840, surface 832 or surface 834 may equally as readily be used to determine the trajectory 840 in a manner similar to that described below for surface 833. Additionally, the space between the two surfaces 832, 834 may be partly or completely filled with a radiolucent material 853, such as a resin foam or the like, that can enhance the structural rigidity of the apparatus 830 and further help steady the instrument 828. This is illustrated in broken lines in FIG. 12.

The apparatus 830 is immobilized (that is, fixed to the operating table or any other stationary equipment in the operating room) at a desired location relative to the tissue 822. The anatomy of the tissue 822 is then visualized by using such techniques as x-ray exposures or fluoroscopy to establish the desired trajectory 840. A suitable radiopaque device 828, such as, for example, a K-wire, is then used to locate the points 852, 854 at which the trajectory 840 passes through, for example, the first surface 832 and into the tissue 822. The points 852, 854 are then marked, for example, with a sterile marking pen or by scratching an index with a sterile scribe or the like. The point 854 can be marked directly on the patient. The trajectory between points 852, 854 is the desired trajectory 840 into and through the tissue 822.

Instrument 828 can also be used to form holes at points 852, 854. Instrument 828 can then be advanced, for example, through the intervening tissue of the patient to enter the pedicle 822 along the appropriate trajectory 840. Alternatively, (an)other device(s) can be used to form holes at points 852, 854. A K-wire, for example, can be used to mark the desired trajectory 840 and can then be used to guide other instruments such as a probe, a tap, a drill, a pedicle screw, and so on, along the determined trajectory 840 into the tissue 822.

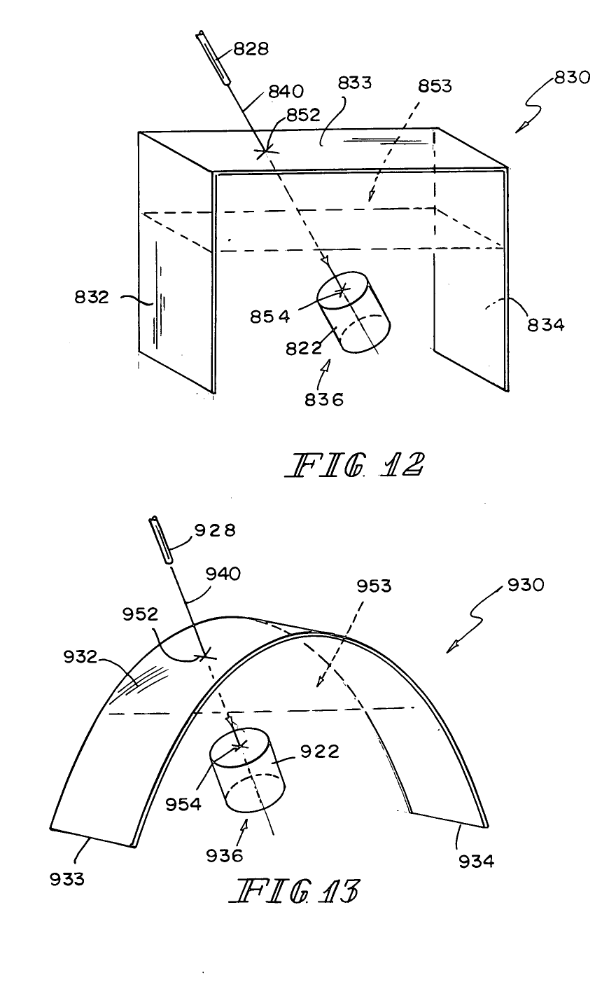

Referring now to FIG. 13, an apparatus 930 comprises a surface 932. The surface 932 is in the form of a section of a right circular cylinder bounded by longitudinal edges 933, 934, and defines a space 936. Insertion of a medical device 928, such as, for example, a K-wire, through the surface 932 and into, for example, a pedicle 922 along a determined trajectory 940 will appropriately locate the medical device 928.

The surface 932 is radiolucent to permit the passage of radiation through the surface 932 and into the patient to permit visualization of the pedicle 922. The surface 932 desirably is also transparent or translucent to permit the passage of light to facilitate determination of the trajectory 940 for insertion of, for example, a K-wire 928 into a pedicle 922 as described in more detail below. Additionally, the space beneath surface 932 may be partly or completely filled with a radiolucent material 953, such as a resin foam or the like, that can enhance the structural rigidity of the apparatus 930 and further help steady the instrument 928. This is illustrated in broken lines in FIG. 13.

The apparatus 930 is immobilized (that is, fixed to the operating table or any other stationary equipment in the operating room) at a desired location relative to the tissue 922. The anatomy of the tissue 922 is then visualized by using such techniques as x-ray exposures or fluoroscopy to establish the desired trajectory 940. A suitable radiopaque device 928, such as, for example, a K-wire, is then used to locate the points 952, 954 at which the trajectory 940 passes through, for example, the surface 932 and into the tissue 922. The points 952, 954 are then marked, for example, with a sterile marking pen or by scratching an index with a sterile scribe or the like. The point 954 can be marked directly on the patient. The trajectory between points 952, 954 is the desired trajectory 940 into and through the tissue 922.

Instrument 928, can also be used to form holes at points 952, 954. Instrument 928 can then be advanced, for example, through the skin and soft tissue of the patient to enter the pedicle 922 along the appropriate trajectory 940. Alternatively, (an)other device(s) can be used to form holes at points 952, 954. A K-wire, for example, can be used to mark the desired trajectory 940 and can then be used to guide other instruments such as a probe, a tap, a drill, a pedicle screw, and so on, along the determined trajectory 940 into the tissue 922.

The method described herein reduces the exposure of both the physician and the patient to radiation because once the proper trajectory for insertion of medical devices, such as probes, taps, drills, screws, pins, and the like, has been determined, fluoroscopy (e.g., using real-time monitoring) or x-ray exposures are no longer necessary to locate the trajectory that leads to the center of the pedicle.

The method described herein can be used to establish a trajectory for insertion of a medical device into a patient for any structure that can be localized by fluoroscopy or x-ray exposures.

Although the illustrated surfaces 32, 34; 132, 134; 232, 234; 332, 334; 432, 434; 532, 534, 632, 634, 732, 734, 832, 833, 834, 932 of the illustrated embodiments are generally planar, this is not a requirement to practice the invention. For example, the “surfaces” may be two points, similar to points 52, 54; 152, 154; 252, 254; 352, 354; 452, 454; 552, 554 on the wall of a cylinder or portion of a cylinder of any suitable cross section perpendicular to the cylinder's axis, such as, for example, two spaced points on a circular cylinder, such as the one illustrated in FIG. 13. The “surfaces” could also be two spaced points, similar to points 52, 54; 152, 154; 252, 254; 352, 354; 452, 454; 552, 554 on a hollow sphere or other hollow solid of rotation or portion of such a hollow solid of rotation, or the like.

The surfaces can also be made of a radiolucent, translucent, rigid mesh, or can be fabricated with one or more holes already formed in them, with such preformed holes to be oriented with points 52, 54; 152, 154; 252, 254; 352, 354; 452, 454; 552, 554; 652; 752; 852; 952 along the desired trajectory 40, 140, 240, 340, 440, 540, 640, 740, 840, 940 through which the K-wire can be placed.

1. An apparatus for determining a trajectory for insertion of a medical device into tissue, the apparatus comprising a first surface which is radiolucent and transparent or translucent and a second surface which is radiolucent and transparent or translucent, the second surface spaced from the first surface so that a point on the first surface and a point on the second surface define the trajectory.

2. The apparatus of claim 1 further comprising means for immobilizing the first and second surfaces relative to the tissue.

3. The apparatus of claim 1 wherein the first and second surfaces are substantially parallel.

4. The apparatus of claim 1 wherein the first and second surfaces are not substantially parallel.

5. The apparatus of claim 1 wherein a distance between the first and second surfaces is adjustable.

6. The apparatus of claim 1 wherein the first and second surfaces are separated by a distance sufficient to permit entry of an instrument between the first and second surfaces to mark at least one of the first and second surfaces.

7. The apparatus of claim 1 wherein the medical device is selected from the group consisting of a wire, a needle, a tap, a screw, a hook, a pin, a staple, a depth gauge, a drill, a drill guide, a probe, a device useful for orthopedic surgical intervention, and combinations of these.

8. The apparatus of claim 1 wherein at least one of the first and second surfaces comprises a plastic.

9. The apparatus of claim 8 wherein the at least one of the first and second surfaces comprises a plastic selected from the group consisting of acrylic, epoxy, polyester, polypropylene, polyurethane, polyethylene, polycarbonate, polystyrene, polysulfone, polyetherimide, polyethersulfone, polyphenylsulfone, polyphenylsulfide, acrylonitrile-butadiene-styrene polymer, polyetheretherketone, and combinations thereof.

10. The apparatus of claim 8 wherein the at least one of the first and second surfaces comprises a filler.

11. A method for determining a trajectory for insertion of a medical device into tissue, the method comprising visualizing the tissue using at least one of x-ray exposures and fluoroscopy, determining the trajectory for insertion of the medical device into the tissue using the apparatus of claim 1 and inserting the medical device into the tissue along the determined trajectory.

12. The method of claim 11 further comprising immobilizing the first and second surfaces relative to the tissue.

13. The method of claim 11 further comprising orienting the second surface at a distance from the first surface.

14. The method of claim 13 wherein orienting the second surface at a distance from the first surface comprises orienting the second surface at a distance from the first surface sufficient to permit entry of an instrument between the first and second surfaces to mark at least one of the first and second surfaces

15. The method of claim 11 wherein determining a trajectory for insertion of a medical device into tissue comprises determining a trajectory for insertion of a medical device selected from the group consisting of a wire, a needle, a tap, a screw, a hook, a pin, a staple, a depth gauge, a drill, a drill guide, a probe, a device useful for orthopedic surgical intervention, and combinations of these, into tissue.

16. A method for determining a trajectory for insertion of a medical device into tissue, the method comprising providing a first surface which is radiolucent and transparent or translucent, providing a second surface which is radiolucent and transparent or translucent, spacing the second surface from the first surface and marking a first point on the first surface and a second point on the second surface to define the trajectory.

17. The method of claim 16 further comprising immobilizing the first and second surfaces relative to the tissue.

18. The method of claim 16 wherein spacing the second surface from the first surface comprises orienting the second surface so that it is substantially parallel with the first surface.

19. The method of claim 16 wherein spacing the second surface from the first surface comprises orienting the second surface so that it is not substantially parallel with the first surface.

20. The method of claim 16 wherein spacing the second surface from the first surface comprises adjustably spacing the second surface from the first surface.

21. The method of claim 16 wherein spacing the second surface from the first surface comprises spacing the second surface from the first surface a distance sufficient to permit entry of an instrument between the first and second surfaces to mark at least one of the first and second surfaces.

22. The method of claim 16 wherein determining a trajectory for insertion of a medical device into tissue comprises determining a trajectory for insertion of a medical device selected from the group consisting of a wire, a needle, a tap, a screw, a hook, a pin, a staple, a depth gauge, a drill, a drill guide, a probe, a device useful for orthopedic surgical intervention, and combinations of these, into tissue.

23. The method of claim 16 wherein providing a first surface which is radiolucent and transparent or translucent and providing a second surface which is radiolucent and transparent or translucent together comprise providing at least one of the first and second surfaces constructed from plastic.

24. The method of claim 23 wherein providing at least one of the first and second surfaces constructed from plastic comprises providing at least one of the first and second surfaces constructed from plastic selected from the group consisting of acrylic, epoxy, polyester, polypropylene, polyurethane, polyethylene, polycarbonate, polystyrene, polysulfone, polyetherimide, polyethersulfone, polyphenylsulfone, polyphenylsulfide, acrylonitrile-butadiene-styrene polymer, polyetheretherketone, and combinations thereof.

25. The method of claim 23 wherein providing at least one of the first and second surfaces constructed from plastic comprises providing at least one of the first and second surfaces constructed from filled plastic.

26. A method for determining a trajectory for insertion of a medical device into a patient, the method comprising providing a surface which is radiolucent and transparent or translucent, spacing a first point on the patient at which the medical device is to be inserted from the surface, and marking the first point on the patient and a second point on the surface to define the trajectory.

27. The method of claim 26 further comprising immobilizing the surface relative to the patient.

28. The method of claim 26 wherein determining a trajectory for insertion of the medical device into the patient comprises determining a trajectory for insertion of a medical device selected from the group consisting of a wire, a needle, a tap, a screw, a hook, a pin, a staple, a depth gauge, a drill, a drill guide, a probe, a device useful for orthopedic surgical intervention, and combinations of these, into the tissue.

29. The method of claim 26 wherein providing a surface which is radiolucent and transparent or translucent comprises providing the surface constructed from plastic.

30. The method of claim 29 wherein providing the surface constructed from plastic comprises providing the surface constructed from plastic selected from the group consisting of acrylic, epoxy, polyester, polypropylene, polyurethane, polyethylene, polycarbonate, polystyrene, polysulfone, polyetherimide, polyethersulfone, polyphenylsulfone, polyphenylsulfide, acrylonitrile-butadiene-styrene polymer, polyetheretherketone, and combinations thereof.

31. The method of claim 29 wherein providing the surface constructed from plastic comprises providing the surface constructed from filled plastic.

32. An apparatus for determining a trajectory for insertion of a medical device into a patient, the apparatus comprising a surface which is radiolucent and transparent or translucent, and means for immobilizing the surface relative to the patient, the surface spaced from the patient so that a point on the surface and a point on the patient define the trajectory for insertion of the medical device into the patient.

33. The apparatus of claim 32 wherein the medical device is selected from the group consisting of a wire, a needle, a tap, a screw, a hook, a pin, a staple, a depth gauge, a drill, a drill guide, a probe, a device useful for orthopedic surgical intervention, and combinations of these.

34. The apparatus of claim 32 wherein the surface comprises a plastic.

35. The apparatus of claim 32 wherein the surface comprises a plastic selected from the group consisting of acrylic, epoxy, polyester, polypropylene, polyurethane, polyethylene, polycarbonate, polystyrene, polysulfone, polyetherimide, polyethersulfone, polyphenylsulfone, polyphenylsulfide, acrylonitrile-butadiene-styrene polymer, polyetheretherketone, and combinations thereof.

36. The apparatus of claim 32 wherein the surface comprises a filled plastic.
